(19) United States
(12) Patent Application Publication
(10) Pub. No.: US 2026/0245700 A1
(43) Pub. Date: Aug. 20, 2026

(54) METHOD FOR GIVING A RECOMMENDATION ON THE DIETARY ENERGY OF A POULTRY DIET

(71) Applicant: Evonik Operations GmbH, Essen (DE)

(72) Inventors: Juliano Cesar DE PAULA DORIGAM, Hanau (DE); Lukas BAUER, Dammbach (DE); Martina KLÜNEMANN, Frankfurt (DE); Andreas LEMME, Mömbris (DE)

(73) Assignee: Evonik Operations GmbH, Essen (DE)

(21) Appl. No.: 19/159,872

(22) PCT Filed: Mar. 5, 2024
(86) PCT No.: PCT/EP2024/055728

§ 371 Date: Aug. 26, 2025

(30) Foreign Application Priority Data
Mar. 7, 2023 (EP) 23160442.2

(51) Int. Cl. G16H 20/60; A01K 45/00; G16H 10/60

(52) U.S. Cl. CPC G16H 20/60; A01K 45/00; G16H 10/60

(57) ABSTRACT

The present invention relates to a computer-implemented method for giving a recommendation on the dietary energy level of a poultry diet, comprising the steps of a) receiving, requesting, and/or providing data from the input/output device of a user, b) determining the actual poultry energy required E_{required}, c) determining the relation between the dietary energy level E_{CD} of step a) and the actual poultry energy required E_{required} of step b), d) determining, for the poultry sex and the day of the feeding phase of step a), the energy reduction E_{reduction}, and e) sending the energy reduction E_{reduction} determined to the input/output device of step a).

The present invention relates to a computer-implemented method for giving a recommendation on the dietary energy level of a poultry diet and a system for giving a recommendation on the dietary energy level of a poultry diet.

It is a challenge for everyone involved in poultry production to feed birds with a balanced diet, at the lowest cost, yet still produce eggs and meat that meet the user's requirements and attract premium prices to maximize profit, all while meeting environmental and animal welfare requirements.

Commercial feed manufacturers and farmers have faced this challenge for many years as they seek to reduce the cost of feed without compromising the birds' productivity. This is an important consideration as feed constitutes the highest variable cost in poultry production, according for at least 70% of the total production cost. Energy is already the most expensive nutrient when formulating poultry diets and that is unlikely to change given the stiff competition for available energy sources for human food. This means energy feed ingredients are becoming scarce and expensive for use in poultry production.

Published patent application US 2018/0350010 A1 discloses a method and system in an adaptive framework for modeling livestock growth. The adaptive framework processes input data relative to livestock growth in an ensemble of one or more models and an artificial intelligence layer configured to select the most appropriate or primary model to optimize, predict, and recommend livestock feed operations based upon environmental, physiological, location, and time variables within such input data. The adaptive framework also optimizes workflow by pen and by producer, based upon historical performance, gender and breed and the management practices of the producer.

Published patent application US 2021/0241880 A1 discloses a computer-implemented methods for the determination and/or assessment of processing influences on the energy value of a feedstuff raw material and/or feedstuff, in which the processing conditions indicator of the feedstuff raw material and/or feedstuff is determined and the corrected energy values of the feedstuff raw material and/or feedstuff are determined.

Published patent application WO 2022/204656 A1 discloses various approaches for determining an animal feed formulation. In some implementations, a system may obtain data that provides characteristics of an animal population located at a site. The system may identify a target growth schedule for the animal population, using the target growth schedule and the characteristics of the animal population, as the model predicts nutrition requirements based on projected nitrogenous energy requirements for the tissue growth. The system may identify a feed formulation producible from a combination of animal feed ingredients to satisfy the nutrition requirements for tissue growth. The system may generate a data output for sue of the feed formulation with the animal population, the data output indicating the identified feed formulation producible from the combination animal feed ingredients.

Published patent application WO 2022/238351 A1 discloses a precision feed formulation. It further relates to a computer-implemented method of predicting the impact of corn quality in an animal feed on animal feed conversion ratio. It further relates to the use of a model for improving feed conversion ratio.

Published patent application US 2007/0026493 A1 discloses a system for generating optimized values for variable inputs to an animal production system. The system includes a simulator engine configured to receive a plurality of animal information inputs and generate a performance projection. At least one of the animal information inputs is designated as a variable input and at least one of the animal information inputs includes animal genotype information. The system further includes an enterprise supervisor engine configured to generate an optimized value for at least one variable input wherein the optimized value is configured to optimize animal production based on the animal genotype information.

Feed intake and feed efficiency in poultry is regulated, at least in part, by ME levels. Fast-growing animals, such as broilers, pigs, and layers, require substantial amounts of energy to grow muscle tissue. All animals get the energy in their cells from adenosine triphosphate (ATP). The amount of ATP in cells is strictly regulated, and it is only available for a short period of time; characterized by a high need in energy.

Creatine (CREA) plays a key role in energy balance in muscle cells. The system “creatine phosphate/CREA” acts as a buffer, guaranteeing the permanent availability of ATP molecules. Creatine phosphate (PCREA) is a dynamic storage of energy-rich phosphate and ensures a stable supply of ATP/ADP in the cell.

Guanidinoacetic acid (GAA) is a stable and widely available feed supplement that is mainly used to boost energy efficiency. It is efficiently converted to CREA in the body. CREA and its phosphorylated form PCREA play a crucial role in cellular energy metabolism. Though CREA is naturally synthesized in the body of all vertebrates (Daly MM. (1985) Guanidinoacetate methyltransferase activity in tissues and cultured cells. Arch Biochem Biophys. 1; 236(2):576-84. doi: 10.1016/0003-9861(85)90661-7. PMID: 3970526; Stead, L., M., K. P. Au, R. L. Jacobs, M. L. Bronson, and E. T. Brosnan (2001) “Methylation Demand and Homocysteine Metabolism: Effects of Dietary Provision of Creatine and Guanidinoacetate.” American Journal of Physiology Endocrinology and Metabolism 281: 1095-1100. doi:10.1152/ajpendo.2001.281.5.E1095; Komoto, J., Y. Takata, T. Yamada, K. Konishi, H. Ogawa, T. Gomi, M. Fuioka, and F. Takusagawa (2003) “Monoclinic Guanidinoacetate Methyltransferase and Gadolinium Ion-binding Characteristics.” Acta Crystallographia D 59: 1589-1596. doi:10.1107/S0907444903014719), its endogenous synthesis may not be sufficient to support the extraordinary growth rate resulting from modern poultry genetics. Nowadays, poultry diets are mainly composed of ingredients from plant origin, mainly corn and soybean meal. CREA is only found in feed ingredients of animal origin, while plant-based ingredients do not contain any metabolites of CREA (Krueger, K., K. Damme, and A. Lemme (2010) “Bessere Mast mit CreAmino.” DGS Magazin 26/2010, 10-14).

GAA, a direct precursor of CREA, has attracted attention as a feed additive because of its high thermal stability (Vranes, M., S. Ostojic, A. Tot, S. Papovic, and S. Gadzuric (2017) “Experimental and Computational Study of GAA Self-aggregation in Aqueous Solution.” Food Chemistry 237: 53-57. doi:10.1016/j.foodchem.2017.05.088), which makes it relatively stable during pelleting or extruding. GAA has a high bioavailability (EFSA (European Food Safety Authority) 2009). “Safety and Efficiency of Guanidino Acetic Acid as Feed Additive for Chickens for Fattening.” The EFSA Journal 988: 1-30; Tossenberger, J., M. Rademacher, K. Nemeth, V. Halas, and A. Lemme. 2016. “Digestibility and Metabolism of Dietary Guanidino Acetic Acid Fed to Broilers.” Poultry Science 95: 2058-2067. doi:10.3382/ps/pew083) and is cost-effective with many diet compositions. Accordingly, GAA has been officially registered as an animal feed additive by American and European regulatory agencies (FDA Federal Register 81, 30 Nov. 2016; EUR-Lex, L270/4, 5 Oct. 2016).

When GAA is supplemented in broiler diets, muscle CREA increases asymptotically up to a maximum of 29% compared to non-supplemented diets. Supplementation of 0.6 to 1.2 g/kg, in the range of applicable GAA supplementation by the European Union (EFSA 2009), would increase muscle CREA by 14 to 21% (see FIG. 1). Muscle CREA levels in non-supplemented broilers are reported to be between 3986 and 4789 mg/kg breast meat (Lemme, A., J. Ringel, H. S. Rostagno, and M. S. Redshaw (2007) “Supplemental Guanidine Acetic Acid Improved Feed Conversion, Weight Gain and Breast Meat Yield in Male and Female Broilers.” In Proceedings of 16th European Symposium on Poultry Nutrition, Strasbourg, France, 335-338; Ringel, J., A. Lemme, A. Knox, J. Mc Nab, and M. S. Redshaw (2007) “Effects of Graded Levels of Creatine and Guanidine Acetic Acid in Vegetable Based Diets on Performance and Biochemical Parameters in Muscle Tissue.” In Proceedings of 16th European Symposium on Poultry Nutrition, Strasbourg, France, 387-390. doi:10.1016/j.ejpb.2007.03.018; Majdeddin, M., A. Golian, H. Kermanshahi, S. de Smet, and J. Michiels (2018) “Guanidinoacetic Acid Supplementation in Broiler Chickens Fed on Corn-soybean Diets Affects Performance in the Finisher Period and Energy Metabolites in Breast Muscle Independent of Diet Nutrient Density.” British Poultry Science 59: 443-451. doi:10.1080/00071668.2018.1476678; Tossenberger, J., M. Rademacher, K. Nemeth, V. Halas, and A. Lemme. 2016. “Digestibility and Metabolism of Dietary Guanidino Acetic Acid Fed to Broilers.” Poultry Science 95: 2058-2067. doi:10.3382/ps/pew083).

The effect of GAA on performance and metabolism is particularly well-understood in broilers. GAA positively influences feed conversion ratio (FCR), body weight gain (BWG) and breast meat yield in broilers (McBreairty L. E., Robinson J. L., Furlong K. R., Brunton J. A., Bertolo R. F. (2015) Guanidinoacetate Is More Effective than Creatine at Enhancing Tissue Creatine Stores while Consequently Limiting Methionine Availability in Yucatan Miniature Pigs. PLoS ONE 10(6): e0131563. https://doi.org/10.1371/journal.pone.0131563; De Groote, A. A. (2015) “Efficacy of Dietary Guanidinoacetic Acid in Broiler Chicks.” Master Degree. Urbana IL: University of Illinois at Urbana-Champaign; M.

Majdeddin, U. Braun, A. Lemme, A. Golian, H. Kermanshahi, S. De Smet, J. Michiels (2020) «Guanidinoacetic acid supplementation improves feed conversion in broilers subjected to heat stress associated with muscle creatine loading and arginine sparing.»Poultry Science, Volume 99, Issue 9, 2020, Pages 4442-4453, ISSN 0032-5791, https://doi.org/10.1016/j.psj.2020.05.023; Zhang B, Liu N, He Z, Song P, Hao M, Xie Y, Li J, Liu R and Sun Z (2021) “Guanidino-Acetic Acid: A Scarce Substance in Biomass That Can Regulate Postmortem Meat Glycolysis of Broilers Subjected to Pre-slaughter Transportation.” Front. Bioeng. Biotechnol. 8:631194. doi: 10.3389/fbioe.2020.631194; Khajali et al., World's Poultry Science Journal, 2020).

The most consistent effect of supplemental GAA was found for FCR. The FCR data from available trials can be transformed to a relative scale, where the un-supplemented control treatment was always set at 100% (see FIG. 2). With only one exceptional data point, GAA supplementation improved FCR in a dose dependent and rather linear manner. According to linear regression, FCR in GAA doses between 0.6 and 1.2 g/kg suggested an improvement by between 4.5 and 8.8 points, respectively. Variation of these 32 data sets is reasonable (R²=0.56). Despite the attempt to normalize data using a relative scale, such variation can be explained by difference in the trial set-up (F. Khajali, A. Lemme & M. Rademacher Heilshorn (2020) “Guanidinoacetic acid as a feed supplement for poultry.” World's Poultry Science Journal, DOI: 10.1080/00439339.2020.1716651).

While the trend to FCR is obvious, results of the meta-analysis for BWG and breast meat yield were not as consistent with an R² of only 0.28 and 0.33, respectively. However, the regression slopes were positive, which suggests that GAA had a positive impact rather than non or a negative influence with increasing supplementation rates (F. Khajali, A. Lemme & M. Rademacher Heilshorn (2020) “Guanidinoacetic acid as a feed supplement for poultry.” World's Poultry Science Journal, DOI: 10.1080/00439339.2020.1716651).

Additionally, to its effects on performance when used on top of un-supplemented diets, several reports suggested that supplemental GAA can reduce ARG requirements of broiler chickens (Dilger, R. N., K. Bryant-Angeloni, R. L. Payne, A. Lemme, and C. M. Parsons (2013) “Dietary Guanidinoacetic Acid Is an Efficacious Replacement forArginine forYoung Chicks.” Poultry Science 92:171177. doi:10.3382/ps.2012-02425; De Groote, A. A., N. Braun, and R. N. Dilger (2018) “Efficacy of Guanidinoacetic Acid on Growth and Muscle Energy Metabolism in Broiler Chicks Receiving Arginine-deficient Diets.” Poultry Science 97: 890-900. doi:10.3382/ps/pex378; Michiels, J., L. Maertens, J. Buyse, A. Lemme, M. Rademacher, N. A. Dierick, and S. de Smet (2012) “Supplementation of Guanidinoacetic Acid to Broiler Diets: Effects on Performance, Carcass Characteristics, Meat Quality, and Energy Metabolism.” Poultry Science 91: 402-412. doi:10.3382/ps.2011-01585; Ahmadipour, B., F. Khajali, and M. Sharifi (2018c) “Effect of Guanidinoacetic Acid Supplementation on Growth Performance and Gut Morphology in Broiler Chickens.” Poultry Science Journal 6: 1924; Ahmadipour, B., M. Sharifi, and F. Khajali (2018b) “Pulmonary Hypertensive Response of Broiler Chickens to Arginine and Guanidinoacetic Acid under High-altitude Hypoxia.” Acta Veterinaria Hungrica 66: 114124; Ahmadipour, B., S. Naeini, M. Sharifi, and F. Khajali (2018a) “Growth Performance and Right Ventricular Hypertrophy Responses of Broiler Chickens to Guanidinoacetic Acid Supplementation under Hypobaric Hypoxia.” Journal of Poultry Science 55: 6064. doi:10.2141/jpsa.0170044).

This is of practical importance in poultry nutrition, since birds do not possess a functional urea cycle and are entirely dependent on dietary ARG (Khajali, F., and R. F. Wideman (2010) “Dietary Arginine: Metabolic, Environmental, Immunological, and Physiological Interrelationships.” World's Poultry Science Journal 66: 751766. doi:10.1017/S0043933910000711). While common corn-soybean meal diets usually meet the recommended ARG levels, there are some reports that suggested a need for the crystalline form of ARG to be supplemented to broiler chickens under certain circumstances like high altitude (Khajali and Wideman, 2010) and heat stress (Brake, J., and D. Balnave.1995. “Essentiality of Arginine in Broilers during Hot Weather.” In Proceedings of 12th Annual Biokyowa Amino Acid Council Meeting, St Louis, Mo, October 3-5).

Given the biochemical pathway of GAA formation in the kidney, one molecule of ARG and one molecule of glycine (GLY) are required to form one molecule of GAA while releasing one molecule of ornithine. This means that 1.49 g ARG (MW=174.2 g/mol) and 0.64 g GLY (MW=75.1 g/mol) are required for the formation of 1 g GAA (MW=117.1 g) and would indicate that the theoretical ARG-sparing capacity of GGA was 149%. Studies were performed in which ARG was supplemented with or without GAA, enabling estimation of the ARG-sparing activity of GAA. Overall, these feeding studies indicated that GAA has an ARG-sparing potential ranging from 77% to 149% (F. Khajali, A. Lemme & M. Rademacher Heilshorn (2020) “Guanidinoacetic acid as a feed supplement for poultry.” World's Poultry Science Journal, DOI: 10.1080/00439339.2020.1716651). Dilger et al. (2013) found that 3.8 g/kg supplemental L-ARG was needed at a very ARG-deficient diet in the absence of GAA, while only 2.1 g/kg L-ARG was needed at 1.2 g/kg supplemental GAA to reach a given feed conversion ratio. Therefore, 1.7 g/kg L-ARG was compensated by 1.2 g/kg GAA, indicating a replacement efficiency of 142%, which is close to the theoretical value of 149%. However, exceeding ARG requirement of broilers would not allow for further sparing but can still have an impact on performance, which has been attributed to improved energy metabolism.

As mentioned above, dietary GAA increases muscle CREA concentrations, leading to improved energy metabolism in muscle tissues (Lemme, A., C. Elwert, R. Gobbi, and M. Rademacher (2011) “Application of the Guanidino Acetic Acid as Creatine Source in Broilers Fed Diets with or without Fish Meal.” In Proceedings of 18^{th} European Symposium on Poultry Nutrition, Qe§ me, Turkey, 453-455. doi:10.1177/1753193411434038). It can be expected that GAA, as a precursor of CREA, influences energy efficiency, and experiments conducted with poultry have confirmed this expectation.

Reduction of dietary energy content and adding GAA to the feed restored broiler performance, which supports this conclusion (Malins et al., 2017; Mousavi S. N., Afsar A., Lotfollahian H. (2013) Effects of guanidinoacetic acid supplementation to broiler diets with varying energy contents, Journal of Applied Poultry Research, Volume 22, Issue 1, 2013, Pages 47-54, ISSN 1056-6171, https://doi.org/10.3382/japr.2012-00575; Heger, J., J. Zelenka, V. Machander, C. Cruz, M. Lestak, and D. Hampel. (2014) “Effects of Guanidinoacetic Acid Supplementation to Broiler Diets with Varying Energy Content.” Acta Universitatis Agriculturae Silviculturae Mendelianae Brunensis 62: 477-485. doi:10.11118/actaun201462030477; Abudabos, A. M., F. Saleh, A. Lemme, and H. A. H. Zakaria (2014) “The Relationship between Guanidino Acetic Acid and Metabolisable Energy Level of Diets on Performance of Broiler Chickens.” Italian Journal of Animal Science 13: 548-556. doi:10.4081/ijas.2014.3269).

The positive effect of GAA supplementation in broiler diets on performance as well as diet optimization like ARG- and energy-sparing are well reported. However, the magnitude of positive effect varies depending on the supplementation rate, broiler age, ARG- and energy-level in the broiler diet was well as the performance level of the broiler flock. In addition, the profitability of GAA usage is ever changing with high fluctuation in broiler prices and feed costs.

Hence, there is a high risk that GAA is not used in an optimum way in broiler diets due to the complexity and variability of factors influencing the effect of GAA supplementation. Commercial feed manufacturers and farmers, integrators, and farmers strive for maximized return-on-investment when a feed additive like GAA is used in their diets.

Accordingly, there was still a need for a method which gives commercial feed manufacturers and farmers, integrators, and farmers a recommendation on dietary energy- and ARG-level of a poultry diet.

It was found that this object is solved in that different ways are considered how GAA can be efficiently used in broiler operations. In the first step, it is considered how much energy can be spared in different broiler feeding phases by GAA, or in other words it is considered what is the maximum potential of GAA to spare energy in different broiler feeding phases. Based on the obtained results, a recommendation is given to maximize the reduction of energy in feed.

An object of the present invention is therefore a computer-implemented method for giving a recommendation on dietary energy level of a poultry diet, comprising the steps of
a) receiving, requesting, and/or providing data from the input/output device of a user, wherein said data comprise one or more of the poultry and the poultry sex, the dietary energy level E_{CD}, the GAA supplementation rate GAA_{supp.rate}, and a day in a feeding phase,
b) determining the actual poultry energy required E_{required}, comprising the steps of
   b1) pulling one or more matrixes from a database, wherein said one or more matrixes comprise, for each poultry sex and each day in every feeding phase, the actual poultry energy required E_{required}, the body weight BW, the body weight gain BWG, the cumulative feed intake, and the daily feed intake, and
   b2) reading off, for the poultry sex and the feeding phase of step a), the actual energy required E_{required} from the matrix of step b1),
c) determining the relation between the dietary energy level E_{CD} of step a) and the actual poultry energy required E_{required} of step b2), wherein
   when the dietary energy level E_{CD} of step a) is larger than a threshold value for E_{required} of step b2), the method proceeds with step d) to determine the energy reduction E_{reduction},
      or
   when the dietary energy level E_{CD} of step a) is lower than or equal with a threshold value for E_{required} of step b2), the energy reduction E_{reduction} is set to 0, and the method proceeds with step e),
d) determining, for the poultry sex and the day of the feeding phase of step a), the energy reduction E_{reduction}, comprising the steps of
   d1) reading off, for the day of the feeding phase of step a), the body weight BW_{d} and the cumulative feed intake CFI_{d} from the matrix of step b1), and for the day preceding said day the body weight BW_{d-1} and the cumulative feed intake CFI_{d-1} from the matrix of step b1),
   d2) calculating the body weight gain BWG by taking the difference between BW_{d} and BW_{d-1} of step d1) and calculating the daily feed intake FI by taking the difference between CFI_{d} and CFI_{d-1} of step d1),
   d3) calculating, for the day of the feeding phase of step a), the energy efficiency ratio EER by means of the formula

$$EER = \frac{( BWG \times 100 )}{( FI \times ( {E}_{CD} / 1000 ) )} \text{+/-} 5 \%$$

wherein
BWG is the body weight gain of step d2),
FI is the daily feed intake of step d2), and
E_{CD} is the dietary energy level of step a),
d4) calculating the recommended energy E_{recommended} by means of the formula

$${E}_{r ecommended} = BWG \times 100 \times 1000 / EER \times FI$$

wherein
BWG is the body weight gain of step d2),
FI is the daily feed intake of step d2), and
EER is the energy efficiency ratio of step d3),
d5) calculating, for the poultry sex and the feeding phase of step a), the energy reduction E_{reduction} by taking the difference between the dietary energy level E_{CD} of step a) and the E_{recommended} of step d4), and
e) sending the energy reduction E_{reduction} determined in step c) or d5) to the input/output device of step a).

It is preferred that the threshold value for E_{required} of step b2) is in the range of 85 to 95% of E_{required} or in the range of 85 to 90% of E_{required}.

Mainly, the method according to the present invention is intended for use as a decision support, when the effect of GAA supplementation should be optimized in broiler operations. This method is unique in assessing the relevant information from the customer operation, i.e., phase length, performance level, dietary energy, and ARG level. It predicts the potential of GAA to spare energy and ARG in broiler diets as well as giving predictions on additional performance improvements. It supports the use in making decisions on the optimal energy and ARG levels for broiler diets in every single feeding phase.

It was found out that the recommendation for the energy sparing of GAA is not identical in all of the feeding phases. Rather, it was found out that the sparing effect is smaller for younger chicks than for older birds. However, in older birds, it was observed that GAA can spare more energy in finisher diets. It was concluded from these observations that different energy values for different feeding phases is an option for optimization. For this reason, the method according to the present invention considers, where appropriate, specific data for every day in every feeding phase or specific data for every feeding phase.

The energy reduction level as well as the GAA supplementation rate was not always the same in trails in which GAA was used to compensate for energy reduction. In most trials dietary energy level of a standard diet was reduced by 50 kcal/kg feed and 0.06% GAA was supplemented to maintain bird performance. This would be equivalent to an energy-sparing value of 83.333 kcal/kg of GAA. However, considering all 20 identified independent trials in which GAA was used to compensate for dietary energy reduction, a wide range of energy-sparing values from 41.667 to 568.333 kcal/kg was tested. Overall, the trials showed that GAA can compensate more than 83.333 kcal/kg. It can be predicted that an energy-sparing value of 163.889 kcal/kg GAA maintains BWG of the birds, whereas there is still a small improvement for FCR. For FCR, it was found out that even an energy-sparing value of 197.222 kcal/kg GAA maintains performance.

It is preferred that the day in a feeding phase of step a) is the last day in the feeding phase in question.

This has the benefit that said last day gives the length of the feeding phase.

In an embodiment of the method according to the present invention the data received, requested, and/or provided in step a) further comprise the dietary ARG level Arg_{CD}, and the step b2) further comprises reading off, for the poultry sex and the feeding phase of step a), the standard ileal digestible ARG requirement Arg_{requirement} from the matrix of step b1).

In addition to its effects on performance when used on top of un-supplemented diets, several reports suggested that supplemented GAA can reduce ARG requirements of broilers chickens.

ARG is an essential amino acid and has several functions in the metabolism other than serving as a building block for protein. Such functions include serving as the precursor of endogenous nitric oxide which is a potent vasodilator acting via the intracellular second-messenger cGMP (Bode-Böger et al. 1996). ARG is also a precursor of GAA in the metabolism (ARG+GLY=GAA) and is an important substrate for the de-novo synthesis of GAA. However, there is a negative feedback mechanism downregulating the enzyme arginine-glycine-amidino-transferase (AGAT) when concentrations of ornithine (out of GAA formation) and CREA will reach a certain level (Walker 1979). This mechanism avoids maximizing CREA levels in muscle tissue and spares ARG and GLY for other metabolic processes other than the GAA formation. When supplementing GAA in broiler diets, CREA concentrations in the metabolism increase and downregulates the enzyme AGAT as described before which leads to the sparing of ARG (and GLY). Indeed, DeGroot et al. (2018) reported that GAA supplementation increased circulatory ARG levels in birds fed ARG-deficient diets. Therefore, the dietary ARG-level has to be considered when using GAA in diets.

ARG can be a limiting amino acid in low crude protein diets, diets with ingredients low in ARG like wheat, sorghum or DDGS or other factors increasing the ARG requirement such as high altitude, high and cold temperatures. In those cases, GAA has the potential to spare supplemental ARG and the ARG-sparing effect of GAA can be considered.

From a molecular point of view, each kg GuanAMINO® containing 960 g GAA would spare 1428 g ARG (143% of GAA amount) or a supplementation of 600 g GuanAMINO® (576 g GAA) would spare 857 g ARG (0.086% in diet). Several experiments have been carried out to determine the magnitude of the ARG-sparing effect of GAA (Dilger et al., 2013; De Groot et al., 2018; De Groot et al., 2019; Lemme et al., 2018; Emami et al., 2017; Fosoul et al., 2019).

The minimum ARG-sparing effect of 77% or 770 g per kg GuanAMINO® had been determined experimentally with comparing response curves of ARG titrations with and without GuanAMINO® supplementation (Lemme et al., 2018). However, Dilger et al. (2013) followed the same approach but conducted a trial with very deficient ARG diets and reported a ARG-sparing effect of 143% which is in line with the stoichiometric calculation above. Therefore, Khajali et al. (2020) concluded in their review that an ARG-sparing capacity of GAA in chickens can range from 77 to 149%. Khajali et al. (2020) also suggested that in circumstances where spared ARG is used for other purposes (for example, immune response) rather than muscle synthesis, the ARG-sparing effects of GAA may not be observed in growth performance or feed efficiency. Dao and Swick (2021) added that ARG-sparing effects of GAA may not be effective when broilers are fed diets with sufficient levels of ARG and in thermal comfort conditions as described before.

However, when the ARG requirement is exceeded, GAA cannot spare any ARG but can still have an impact on performance, which is then attributed to improved energy metabolism.

In a preferred embodiment the method according to the present invention further comprises the step
d6) determining the ARG sparing potential Arg_{spar.pot}., comprising the steps of
   d6a) calculating the percentage of ARG requirement Arg_{perc. requirement} by means of the formula

$${Arg}_{perc . requirement} [ \% ] - \frac{{Arg}_{CD}}{{Arg}_{requirement}} \times 100 ,$$

d6b) determining the ARG sparing potential Arg_{spar.pot}., wherein
   when the Arg_{perc. requirement} calculated in step d6a) is lower than or equal with 73%, the ARG sparing potential Arg_{spar.pot.} is set to a value of 143%,
   when the Arg_{perc. requirement} calculated in step d6a) is larger than 73% and lower than 100%, the ARG sparing potential Arg_{spar.pot}. is in the range between 143 and 77%, with the endpoints being excluded,
      or
   when the Arg_{perc. requirement} calculated in step d6a) is equal with or greater than 100%, the ARG sparing potential Arg_{spar.pot}. is set to a value of 77%,
      and
d6c) sending the ARG sparing potential Arg_{spar.pot}. determined in step d6b) to the input/output device of step a).

It is preferred that the lower the ARG level in relation to the ARG requirement, the higher is the ARG sparing potential Arg_{spar.pot}., and/or the higher the ARG level in relation to the ARG requirement, the lower is the ARG sparing potential Arg_{spar.pot}.

Considering the different magnitudes of the GAA ARG-sparing effect in the before mentioned trials (Dilger et al., 2013; De Groot et al., 2018; De Groot et al., 2019; Lemme et al., 2018; Emami et al., 2017; Fosoul et al., 2019) and the bigger effect size of GAA at low dietary ARG-levels compared to adequate ARG-levels, the method according to the present invention gives following recommendation:
If the ARG requirement is smaller than or equal as 73%, the method recommends a GAA ARG-sparing potential of 143% (result from Dilger et al., 2013).
If the ARG requirement is greater than 73 and smaller than 100%, then the method recommends an GAA ARG-sparing potential based on following equation:

$${Arg}_{spar . pot .} [ \% ] = 77 + 2.44 \times ( ( {Arg}_{requirement} < 100 ) \times ( 100 - {Arg}_{requirement} )$$

For example, when the ARG requirement is 90%, the method recommends a GAA ARG-sparing potential of 101.4% (=77+2.44×(100−90)).
If the ARG requirement is 100% or more, the method recommends a GAA ARG-sparing potential of 77%.

Before making any further predictions with respect to ARG, the method according to the present invention determines the supplemental amount of ARG Argₛᵤₚₚₗₑₘₑₙₜ required to reach the standard ileal digestible ARG requirement Arg_{requirement}.

In another embodiment the method according to the present invention further comprises the step of
d7) determining the supplemental amount of ARG Argₛᵤₚₚₗₑₘₑₙₜ required to reach the standard ileal digestible ARG requirement Arg_{requirement} by means of the formula

$${Arg}_{supplement} = ( {Arg}_{requirement} - {Arg}_{CD} ) \times \frac{1000}{100} .$$

Using the thus determined supplemental amount of ARG Argₛᵤₚₚₗₑₘₑₙt, the GAA acid supplementation rate GAA_{suppl.rate}, and if necessary, the ARG sparing potential Arg_{spar.pot}., the method according to the present invention makes predictions for the ARG sparing potential with supplemented GAA, Arg_{spar.pot.with suppl.GAA}.

In a further preferred embodiment, the method according to the present invention further comprises the step of
d8) determining the ARG sparing potential with supplemented GAA, Arg_{spar.pot.with suppl.GAA}, wherein
   when the Argₛᵤₚₚₗₑₘₑₙₜ determined in step d7) is lower than 0, the Arg_{spar.pot.with suppl.GAA} is set to a value of 0,
      or
   when the Argₛᵤₚₚₗₑₘₑₙₜ determined in step d7) is equal with or larger than the GAA_{suppl.rate}, the Arg_{spar.pot.with suppl.GAA} is calculated by means of the formula

$${Arg}_{spar . pot . with suppl . GAA} = {Arg}_{spar . pot .} \times {GAA}_{suppl . rate}$$

wherein
   Arg_{spar.pot}. is the ARG sparing potential of step d6b), and
   GAA_{suppl.rate} is the GAA supplementation rate of step 1a).

Using the thus determined ARG sparing potential with supplemental GAA Arg_{spar.pot.with suppl.GAA}, the supplemental amount of ARG Argₛᵤₚₚₗₑₘₑₙₜ required to reach the standard ileal digestibility ARG requirement, the method predicts in the next step the supplemental amount of ARG required Arg_{supplement,required} considering the ARG sparing potential with supplemental GAA Arg_{spar.pot.with suppl.GAA}.

In another preferred embodiment the method according to the present invention further comprises the step of
d9) determining the supplemental amount of ARG required Arg_{supplement,required} considering the ARG sparing potential with supplemental GAA Arg_{spar.pot.with suppl.GAA}, wherein
   when the Argₛᵤₚₚₗₑₘₑₙₜ determined in step d7) is lower than 0, the Arg_{supplement,required} is set to a value of 0,
      or
   when the Argₛᵤₚₚₗₑₘₑₙₜ determined in step d7) is larger than 0, the Arg_{supplement,required} is calculated by taking the difference between the Argₛᵤₚₚₗₑₘₑₙₜ of step d7) and the Arg_{spar.pot.with supp.GAA} of step d8).

In an embodiment of the method according to the present invention the body weight BW and the body weight gain BWG are the standard body weight BW and the standard body weight gain BWG for the poultry and poultry sex of step a).

However, cases are conceivable that neither the body weight BW and the body weight gain BWG nor the standard body weight BW and the standard body weight gain BWG correspond to the desired body weight of the use. The method according to the present invention also considers such a situation in that then the data received, requested, and/or provided in step a) further comprises the target poultry body weight at the day of slaughter BW_{target}.

In another embodiment of the method according to the present invention the data received, requested, and/or provided in step a) further comprises the target poultry body weight at the day of slaughter BW_{target}.

When the target poultry body weight at the day of slaughter BW_{target} differs from the body weight BW or standard body weight BW, first the difference has to be identified as such and next, the parameters determined in any or all of the steps d1) to d9) need to be corrected by the corresponding percentage difference between the target poultry body weight at the day of slaughter BW_{target} and the body weight BW.

In a preferred embodiment of the method according to the present invention the BW_{target} is checked for being identical with or different from the standard body weight BW for the poultry and poultry sex of step a), and when the BW_{target} is different from the standard body weight BW, the percentage difference between BW_{target} and the standard body weight BW is determined, and the parameters determined in any or all of the steps d1) to d9) are corrected by said percentage difference.

In one embodiment of the method according to the present invention each day in the lifetime of each poultry is assigned to a feeding phase of the poultry.

In another embodiment of the method according to the present invention the lifetime of each poultry is divided into individual feeding phases with each feeding having the same or a different length in days.

It was found that data which is relevant for making predictions with the method according to the present invention is not identical for every feeding phase. Even further, it was found that some data which is relevant for making predictions with the method according to the present invention is not even identical for every day in a feeding phase. Therefore, it is beneficial to group that data which is not identical for each sex, and every day within a feeding phase in one separate matrix 1 and the other data, which is different only for each sex and every feeding phase of a poultry into a different matrix.

The data for the body weight BW, the body weight gain BWG, the cumulative feed intake, and the daily feed intake are different for each sex and each day in every feeding phase of a poultry.

Therefore, said data are comprised in a matrix M1.

By comparison, the data for the actual poultry energy required E_{required} and the standard ileal digestible ARG requirement Arg_{requirement} are different only for each sex and every feeding phase of a poultry. Therefore, these data are comprised in a matrix M2.

In another embodiment of the method according to the present invention, for each sex and each day in every feeding phase of a poultry, the body weight BW, the body weight gain BWG, the cumulative feed intake, and the daily feed intake are comprised in a matrix M1.

In a further embodiment of the method according to the present invention, for each sex and every feeding phase of a poultry, the actual poultry energy required E_{required} and the standard ileal digestible ARG requirement Arg_{requirement} are comprised in a matrix M2.

In principle, the method according to the present invention is not subject to any limitation regarding a specific type of poultry. Hence, the method can be used for giving recommendation on the dietary level of the diet for any conceivable type of poultry. In the context of the present invention the term poultry is used as known to the person skilled in the art and denotes any kind of domesticated bird, captive-raised for its utility, and specifically for domesticated birds kept by humans for their eggs their meat or their feathers.

In yet a further embodiment of the method according to the present invention the poultry is broilers, turkeys, ducks, or geese.

Another object of the present invention is a system for giving a recommendation on dietary energy level of a poultry diet, comprising a processing unit adapted to carry out at least the step a) to e) of the method according to the present invention, and having access to the one or more databases of steps b1) and d1).

In an embodiment of the system according to the present invention, the processing unit also comprises the one or more databases of steps b1) and d1).

In another embodiment of the system according to the present invention, the processing unit forms a network with the one or more databases of steps b1) and d1).

A further object of the present invention is a computer-program product comprising instructions which, when the program is executed by a computer, cause the computer to carry out the method according to the present invention.

Yet another object of the present invention is a computer-readable medium comprising instructions which, when executed by a computer, cause the computer to carry out the method according to the present invention.

1. A computer-implemented method for giving a recommendation on dietary energy level of a poultry diet, comprising
a) receiving, requesting, and/or providing data from the input/output device of a user, wherein said data comprise one or more of the poultry and the poultry sex, the dietary energy level E_{CD}, the guanidinoacetic acid (GAA) supplementation rate GAA_{suppl. rate}, and a day in a feeding phase,
b) determining the actual poultry energy required E_{required}, comprising
b1) pulling one or more matrixes from a database, wherein said one or more matrixes comprise, for each poultry sex and each day in every feeding phase, the actual poultry energy required E_{required}, the body weight BW, the body weight gain BWG, the cumulative feed intake. and the daily feed intake, and
b2) reading off, for the poultry sex and the feeding phase of step a), the actual energy required E_{required} from the matrix of step b1),
c) determining the relation between the dietary energy level E_{CD} of step a) and the actual poultry energy required E_{required} of step b2), wherein
when the dietary energy level E_{CD} of step a) is larger than a threshold value for E_{required} of step b2), the method proceeds with step d) to determine the energy reduction E_{reduction},
or
when the dietary energy level E_{CD} of step a) is lower than or equal with a threshold value for E_{required} of step b2), the energy reduction E_{reduction} is set to 0, and the method proceeds with step e),
d) determining, for the poultry sex and the day of the feeding phase of step a), the energy reduction E_{reduction}, comprising
d1) reading off, for the day of the feeding phase of step a), the body weight BW_{d} and the cumulative feed intake CFI_{d} from the matrix of step b1), and for the day preceding said day the body weight BW_{d-1} and the cumulative feed intake CFI_{d-1} from the matrix of step b1),
d2) calculating the body weight gain BWG by taking the difference between BW_{d} and BW_{d-1} of step d1) and calculating the daily feed intake FI by taking the difference between CFI_{d} and CFI_{d-1} of step d1),
d3) calculating, for the day of the feeding phase of step a), the energy efficiency ratio EER by means of the formula
$$EER = \frac{( BWG \times 100 )}{( FI \times ( {E}_{CD} / 1000 ) )} \text{+/-} 5 \%$$
wherein
BWG is the body weight gain of step d2),
FI is the daily feed intake of step d2), and
E_{CD} is the dietary energy level of step a),
d4) calculating the recommended energy E_{recommended} by means of the formula
$${E}_{recommended} = BWG \times 100 \times 1000 / EER \times FI$$
wherein
BWG is the body weight gain of step d2),
FI is the daily feed intake of step d2), and
EER is the energy efficiency ratio of step d3),
d5) calculating, for the poultry sex and the feeding phase of step a), the energy reduction E_{reduction} by taking the difference between the dietary energy level E_{CD} of step a) and the E_{recommended} of step d4), and
e) sending the energy reduction E_{reduction} determined in step c) or d5) to the input/output device of step a).

2. The method according to claim 1, wherein the data received, requested, and/or provided in step a) further comprise the dietary arginine (ARG) level Arg_{CD}, and the step b2) further comprises reading off, for the poultry sex and the feeding phase of step a), the standard ileal digestible ARG requirement Arg_{requirement} from the matrix of step b1).

3. The method according to claim 2, further comprising the step
d6) determining the ARG sparing potential Arg_{spar.pot.}, comprising
d6a) calculating the percentage of ARG requirement Arg_{perc. requirement} by means of the formula
$${Arg}_{perc . requirement} [ \% ] - \frac{{Arg}_{CD}}{{Arg}_{requirement}} \times 100 ,$$
d6b) determining the ARG sparing potential Arg_{spar.pot.}, wherein
when the Arg_{perc. requirement} calculated in step d6a) is lower than or equal with 73%, the ARG sparing potential Arg_{spar.pot.} is set to a value of 143%,
when the Arg_{perc. requirement} calculated in step d6a) is larger than 73% and lower than 100%, the ARG sparing potential Arg_{spar.pot.} is in the range between 143 and 77%, with the endpoints being excluded,
or
when the Arg_{perc. requirement} calculated in step d6a) is equal with or greater than 100%, the ARG sparing potential Arg_{spar.pot.} is set to a value of 77%,
and
d6c) sending the ARG sparing potential Arg_{spar.pot.} determined in step d6b) to the input/output device of step a).

4. The method according to claim 2, further comprising
d7) determining the supplemental amount of ARG Argₛᵤₚₚₗₑₘₑₙₜ required to reach the standard ileal digestible ARG requirement Arg_{requirement} by means of the formula
$${Arg}_{supplement} = ( {Arg}_{requirement} - {Arg}_{CD} ) \times \frac{1000}{100} .$$

5. The method according to claim 4, further comprising
d8) determining the ARG sparing potential with supplemented GAA, Arg_{spar.pot.with suppl.GAA}, wherein
when the Argₛᵤₚₚₗₑₘₑₙₜ determined in step d7) is lower than 0, the Arg_{spar.pot.with suppl.GAA} is set to a value of 0,
or
when the Argₛᵤₚₚₗₑₘₑₙₜ determined in step d7) is equal with or larger than the GAA_{suppl. rate}, the Arg_{spar.pot.with suppl.GAA} is calculated by means of the formula
$${Arg}_{spar . pot . with suppl . GAA} = {Arg}_{spar . pot .} \times {GAA}_{suppl . rate}$$
wherein
Arg_{spar.pot.} is the ARG sparing potential of step d6b), and
GAA_{suppl. rate} is the GAA supplementation rate of step a).

6. The method according to claim 5, further comprising the step of
d9) determining the supplemental amount of ARG required Arg_{supplement, required} considering the ARG sparing potential with supplemental GAA Arg_{spar.pot.with suppl.GAA}, wherein
when the Argₛᵤₚₚₗₑₘₙₑₙₜ determined in step d7) is lower than 0, the Argₛᵤₚₚₗₑₘₑₙₜ, required is set to a value of 0,
or
when the Argₛᵤₚₚₗₑₘₑₙₜ determined in step d7) is larger than 0, the
Arg_{supplement, required} is calculated by taking the difference between the
Argₛᵤₚₚₗₑₘₑₙₜ of step d7) and the Arg_{spar.pot. with suppl. GAA} of step d8).

7. The method according to claim 1, wherein the body weight BW and the body weight gain BWG are the standard body weight BW and the standard body weight gain BWG for the poultry and poultry sex of step a).

8. The method according to claim 1, wherein the data received, requested, and/or provided in step a) further comprises the target poultry body weight at the day of slaughter BW_{target}.

9. The method according to claim 8, wherein the BW_{target} is checked for being identical with or different from the standard body weight BW for the poultry and poultry sex of step a), and when the BW_{target} is different from the standard body weight BW, the percentage difference between BW_{target} and the standard body weight BW is determined, and the parameters determined in any or all of the steps d1) to d9) are corrected by said percentage difference.

10. The method according to claim 1, wherein each day in the lifetime of each poultry is assigned to a feeding phase of the poultry.

11. The method according to claim 1, wherein the lifetime of each poultry is divided into individual feeding phases with each feeding having the same or a different length in days.

12. The method according to claim 1, wherein, for each sex and each day in every feeding phase of a poultry, the body weight BW, the body weight gain BWG, the cumulative feed intake, and the daily feed intake are comprised in a matrix M1.

13. The method according to claim 1, wherein, for each sex and every feeding phase of a poultry. the actual poultry energy required E_{required} and the standard ileal digestible ARG requirement Arg_{requirement} are comprised in a matrix M2.

14. A system for giving a recommendation on dietary energy level of a poultry diet, comprising a processing unit adapted to carry out at least the step a) to e) of the method according to claim 1, and having access to the one or more databases of steps b1) and d1).

15. A computer-program product comprising instructions which, when the program is executed by a computer, cause the computer to carry out the method according to claim 1.

16. A computer-readable medium comprising instructions which, when executed by a computer, cause the computer to carry out the method according to claim 1.
